(19) United States
(12) Patent Application Publication
(10) Pub. No.: US 2024/0268752 A1
(43) Pub. Date: Aug. 15, 2024

(54) BURN INJURY MANAGEMENT USING A CALCULATION OF TOTAL BODY SURFACE AREA BURNED

(71) Applicant: CHILDREN'S NATIONAL MEDICAL CENTER, Washington, DC (US)

(72) Inventor: Cindy COLSON, Washington, DC (US)

(73) Assignee: CHILDREN'S NATIONAL MEDICAL CENTER, Washington, DC (US)

(21) Appl. No.: 18/561,080

(22) PCT Filed: Jun. 9, 2022
(86) PCT No.: PCT/US2022/032829

§ 371 Date: Nov. 15, 2023

Related U.S. Application Data
(60) Provisional application No. 63/208,936, filed on Jun. 9, 2021.

(51) Int. Cl. A61B 5/00; G06T 7/00; G06T 7/62; G16H 70/60

(52) U.S. Cl. CPC A61B 5/445; G06T 7/0012; G06T 7/62; G16H 70/60; G06T 2207/30088

(57) ABSTRACT

A method for determining a burn percentage of a patient, including presenting, via processing circuitry, an image of at least one segment of a body, receiving, via the processing circuitry, a burn indication representing areas within the at least one segment of the body that have sustained a burn, determining, via the processing circuitry, a burned percentage of the at least one segment of the body based on the burn indication, determining, via the processing circuitry, a surface area of the at least one segment of the body based on an age of the patient and a weight of the patient, and determining, via the processing circuitry, the burn percentage of the patient based on the burned percentage of the at least one segment of the body and the surface area of the at least one segment of the body.

### CROSS-REFERENCE TO RELATED APPLICATIONS

The present application claims priority to U.S. Provisional Application No. 63/208,936, filed Jun. 9, 2021, which is incorporated herein by reference in its entirety for all purposes.

### BACKGROUND

### Field of the Disclosure

The present disclosure relates to burn injury assessment and management.

### Description of the Related Art

A burn is the partial or complete destruction of tissue, such as skin, by some form of energy, such as thermal energy, radiation, chemical contact, or electrical contact. Burns can range from minor harm to life-threatening emergencies requiring immediate medical attention. The treatment and management of burns depend on the location and severity of the tissue damage. Burn severity can be quantified by a percentage of total body surface area (TBSA) affected. Burn TBSA can be used to determine a level of care and/or a type of burn treatment approach, such as intravenous fluid resuscitation, wound dressings, therapy, or surgery.

Initial estimates of burn TBSA are often obtained by emergency medicine providers using visual assessment. These initial estimates guide further decisions to transfer patients to designated burn treatment centers and to administer treatment such as fluid resuscitation. In smaller burns, differences in burn TBSA of as little as 5% can seriously affect a disposition of a patient and can lead to a patient being transferred from a floor unit to intensive care. Underestimation of burn size can delay transfers and appropriate treatment in the early resuscitation period. Overestimation of burn size can result in inappropriate burn center treatment, which is associated with healthcare costs in excess of $250 million in the United States each year. Conventional methods and frameworks for estimating burn TBSA can result in overestimation by as much as three-fold, especially for pediatric patients. Additionally, conventional methods for estimating burn TBSA are subjective and can require human computation, which can result in errors during emergency situations.

The foregoing Background description is for the purpose of generally presenting the context of the disclosure. Work of the inventors, to the extent it is described in this background section, as well as aspects of the description which may not otherwise qualify as prior art at the time of the filing, are neither expressly or impliedly admitted as prior art against the present disclosure.

### SUMMARY

According to one embodiment, the present disclosure relates to a method for determining a burn percentage of a patient, including presenting, via processing circuitry, an image of at least one segment of a body, receiving, via the processing circuitry, a burn indication representing areas within the at least one segment of the body that have sustained a burn, determining, via the processing circuitry, a burned percentage of the at least one segment of the body based on the burn indication, determining, via the processing circuitry, a percentage of TBSA of the at least one segment of the body based on an age of the patient and a weight of the patient, and determining, via the processing circuitry, the burn percentage of TBSA of the patient based on the burned percentage of the at least one segment of the body and the percentage of TBSA of the at least one segment of the body.

According to one embodiment, the present disclosure relates to an apparatus for determining a burn percentage of a patient, including processing circuitry configured to present an image of at least one segment of a body, receive a burn indication representing areas within the at least one segment of the body that have sustained a burn, determine a burned percentage of the at least one segment of the body based on the burn indication, determine a surface area of the at least one segment of the body based on an age of the patient and a weight of the patient, and determine the burn percentage of the patient based on the burned percentage of the at least one segment of the body and the surface area of the at least one segment of the body.

According to one embodiment, the present disclosure relates to a non-transitory computer-readable storage medium for storing computer-readable instructions that, when executed by a computer, cause the computer to perform a method for determining a burn percentage of a patient, the method including presenting, via processing circuitry, an image of at least one segment of a body, receiving, via the processing circuitry, a burn indication representing areas within the at least one segment of the body that have sustained a burn, determining, via the processing circuitry, a burned percentage of the at least one segment of the body based on the burn indication, determining, via the processing circuitry, a surface area of the at least one segment of the body based on an age of the patient and a weight of the patient, and determining, via the processing circuitry, the burn percentage of the patient based on the burned percentage of the at least one segment of the body and the surface area of the at least one segment of the body.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 illustrates a method for evaluating burns, according to one embodiment of the present disclosure;

FIG. 2 illustrates a shaded segment of the body representing a burn, according to one embodiment of the present disclosure;

FIG. 3 illustrates shaded segments of the body representing burns, according to one embodiment of the present disclosure;

FIG. 4 illustrates an example assessment of burns, according to one embodiment of the present disclosure;

FIG. 5 illustrates error margins for various methods of evaluating burns, according to one embodiment of the present disclosure;

FIG. 6 is a schematic of a user device for performing a method, according to one embodiment of the present disclosure;

FIG. 7 is a schematic of a hardware system for performing a method, according to one embodiment of the present disclosure; and

FIG. 8 is a schematic of a hardware configuration of a device for performing a method, according to one embodiment of the present disclosure.

### DETAILED DESCRIPTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

Conventional methods for estimating and quantifying burn TBSA include the Lund-Browder Chart, the Rule of Nines, and the Rule of Palms. The Lund-Browder Chart presents a full anterior view and a full posterior view of the human body and assigns size percentages to various sections of the body, such as the right upper arm, right thigh, anterior trunk, etc. Providers can estimate a burn TSBA by estimating the burned percentage of each section and the chart's estimation of the percentage of total body surface area that the section comprises. The Lund-Browder Chart typically includes six age categories for patients ranging from infants (0 to 1 years old) to adults. Providers, especially non-specialists, find the Lund-Browder method challenging because of the need to estimate percentages of burned areas. In addition, the presentation of the full anterior view and the full posterior view of the human body can make it difficult to accurately assess burns in different regions of the body, especially when the burns are small (e.g., less than 20% TBSA). To address limitations of the Lund-Browder method, the Rule of Nines requires similar estimation of burned percentages of sections of the body representing multiples of 9% of body surface area. For example, the torso may be estimated to be 18% of an adult body surface area, a head and neck may be 9%, etc. Disadvantageously, the Rule of Nines distribution for adults does not apply to children or infants, and providers must use different percentages when assessing younger patients. In addition, the Rule of Nines also depends on estimation on a full anterior and/or posterior view of the body.

The Rule of Palms uses the size of the patient's hand as a reference for burn size, with the assumption that the hand (palm and fingers) of a patient makes up approximately 1% of total body surface area. The Rule of Palms can be applied to patients of various ages and sizes because the patients themselves are used as references. However, measuring burns using the size of the hand can lead to inaccuracies due to the shape of the hand and differences in perception when viewing the full body. Computer-based or digital applications for estimating burn TBSA can use methods such as the Lund-Browder Chart, the Rule of Nines, and the Rule of Palms. These digital applications require providers to indicate patient burns on a full posterior and/or full anterior view of the human body. Patient information such as age can be used to estimate burn TBSA based on the categories of the Lund-Browder chart. However, shading in burn areas on a full view of the human body can be challenging, especially for burns that are non-uniform, small, and/or disperse across the body. Precise and accurate shading of burn areas on a full view of the human body can also be difficult in emergency situations when using a mobile device such as a smartphone or a tablet. Zooming in on regions of the body from a full view can also distort a user's perspective of body parts relative to the entire body.

Several factors contribute to discrepancies in burn TBSA estimation, including the depth of the burn injury, the method of estimation used, and the subjectivity of estimation. Estimates of burn TBSA obtained using existing tools and methods vary between burn care specialists and are less accurate when performed by those without specific burn training. Estimation of burn size is needed in prehospital settings and at non-burn hospitals to determine whether patients need to be triaged to a specialty burn center. In addition, initial resuscitation is based on burn size, with inaccurate estimation of this value contributing to over- and under-resuscitation.

Due to inaccurate burn TBSA estimation, patients can experience significant morbidity and mortality. Up to 30% of patients referred to a burn center are under-resuscitated and more than 45% are over-resuscitated, with some receiving as much as 145% of fluid requirements. Under-resuscitation of a burn patient can lead to inadequate fluid administration and delayed wound healing, while over-resuscitation can lead to compartment syndromes, acute respiratory distress syndrome, prolonged ventilator dependence, and increased mortality. Overestimation of burn TBSA is especially prevalent in infants and children because of how rapidly their bodies grow. The body surface area percentages of segments of the body are different in an adult body compared to a child body, and these differences must be considered in estimating burn TBSA.

Estimation of burn TBSA can be most important for burns that take up less than 50% of TBSA in order to determine what type of medical care a patient should receive and whether they should be transferred to a specialized burn treatment center. Therefore, there is a need for methods for estimation of smaller burns that can yield accurate and consistent results. In addition, automated calculations of burn TBSA can reduce human error and improve the process of assessing burn patients.

In one embodiment, the present disclosure can relate to systems and methods for accurate estimation of burn TBSA using digital input of burn information. In one embodiment, the systems and methods can be implemented in an application or a web application accessible by a mobile device, such as a smartphone or tablet. The method in one embodiment can include presenting individual segments of the body for digital input of burn information. The percentage of total body surface area for each segment can be known using an algorithm or a look-up table. Digital burn information for each segment can be collected. The digital burn information for each segment can then be aggregated to determine a burn TBSA.

In one embodiment, the systems and methods can use patient information such as age and weight to more accurately estimate a patient's burn TBSA. The distribution of the body's surface area in infants and children can change dramatically as they age and is different from that of an adult. Age and weight are typically easily obtainable metrics in an emergency medicine situation. For example, age and weight may be recorded during patient intake in an emergency room. If weight is not readily available, alternative methods such as the Broselow Chart can be used to predict a child's weight based on their age and length when lying down. Weight is an important factor in estimating body size and thus body surface area. Most conventional methods, including the Lund-Browder Chart and the Rule of Nines, only use age ranges and do not use weight to determine differences in body sizes when estimating burn TBSA.

In one embodiment, a patient's age in months can be used to estimate burn TBSA. A difference in age of 1-2 months can result in a different distribution of total body surface area across various body parts, especially for infant patients. For example, an infant's head is relatively large compared to the body and takes up a larger percentage of total body surface area than an adult's head. This percentage decreases as the infant grows through childhood. The present method can factor in these differences to estimate burn TBSA. Thus, the method can accurately estimate burn TBSA even for infant patients, who grow at exponential rates. In one embodiment, the burn TBSA can be estimated without the patient's height. Many metrics use the height of the patient to estimate total body surface area. However, height of a patient may be difficult to measure if a patient is unconscious or otherwise immobile. Measuring height can take additional time in an emergency situation and misreporting a patient's height can lead to inaccurate estimations of body surface area. Therefore, certain embodiments can present an advantage in not requiring the height of the patient in determining burn TBSA.

According to one embodiment, the body can be divided into segments to determine burn TBSA, wherein each segment can comprise a known percentage, proportion, or other metric of surface area. In one embodiment, each segment can comprise a known percentage of the total body surface area. Various segments and number of segments can be compatible. For example, the body can be divided into approximately 15-50 segments. As a non-limiting example, the body can be divided into the head, neck, upper arms, lower arms, hands, chest abdomen, back, genitalia, buttocks, thighs, lower legs, feet, etc. In one embodiment, the segments of the body can be portions of the body as divided by anatomical planes, including, but not limited to, a coronal plane, a sagittal plane, and/or a transverse (axial) plane. For example, an arm can be divided into the anterior (front) of the arm as a first segment and the posterior (back) of the arm as a second segment. The division of the segments by anatomical planes can allow collection of burn information with greater detail and accuracy. Segmenting the same body part (e.g., the arm) into multiple views can allow the user to assess and represent a burn that wraps circumferentially around the arm with greater accuracy. The segments thus cover all of the body surface area in a three-dimensional view. As a non-limiting example, the segments of the body can include head anterior, head posterior, neck anterior, neck posterior, right hand anterior, right hand posterior, left hand anterior, left hand posterior, right upper arm anterior, right upper arm posterior, right lower arm anterior, right lower arm posterior, left upper arm anterior, left upper arm posterior, left lower arm anterior, left lower arm posterior, chest-abdomen, back, genitalia, buttocks, left thigh anterior, left thigh posterior, left lower leg anterior, left lower leg posterior, right thigh anterior, right thigh posterior, right lower leg anterior, right lower leg posterior, right foot anterior, right foot posterior, left foot anterior, and left foot posterior. It can be appreciated that segments of the body are not limited to anatomical parts of the body but can include alternate groupings of body parts and smaller or larger regions of the body. For example, the segments can be based on common burn patterns. In one embodiment, the segments of the body and the number of segments can vary depending on the age and weight of the patient. In another embodiment, the segments and the number of segments can be selected by a user.

Each segment of the body comprises a percentage of total body surface area. The percentage can depend on the age and weight of the patient. For example, the thighs and legs take up a percentage of the total body surface area. This percentage changes every 2-4 months for infants up until the age of two, and then approximately every year for children until adolescence. Meanwhile, other anatomical areas, including, but not limited to, the neck, arms, hands, chest/abdomen, back, genitalia, buttocks, and feet may take up an approximately constant percentage of total body surface area throughout development.

In one embodiment, the systems and methods for determining burn TBSA can include a plurality of look-up tables, wherein each look-up table includes the distribution of total body surface area by body segment for a given combination of age and weight. For example, each body segment can make up a percentage of total body surface area. The look-up table can be a unique combination of a body mapping chart with an age and weight. In one embodiment, each look-up table can correspond to a range of age and weight values. For example, a look-up table can include the body surface area for each segment of the body for a 2-month old infant weighing between 0-5 kg. Another look-up table can include the percentage of total body surface area for each segment of the body for an older infant (e.g., 3 months old) within the same weight range. The look-up table can include accurate distributions of body surface area based on weight and age in months. The percentages of all of the segments of the body for a given look-up table can sum to 100% to cover the entire body. In one embodiment, the values in the look-up tables can be calculated using three-dimensional (3D) body scans. According to another embodiment, the values in the look-up tables can be determined and/or validated using clinical data. In one embodiment, the look-up tables can be created using an automated process, e.g., a machine learning network that uses clinical data to determine percentage distributions of body surface area based on age and weight. In one embodiment, an algorithm or calculation method can be used in addition to or in place of a look-up table to accurately determine the percentage of total body surface area of each segment of the body for a given combination of age and weight.

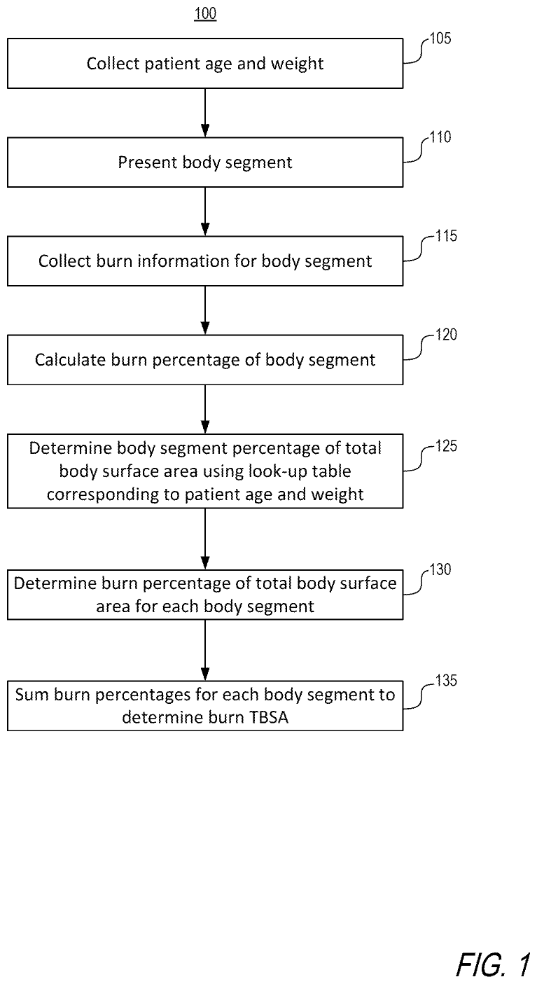

FIG. 1 illustrates a method 100 for determining burn TBSA according to one embodiment. The method 100 can be executed using an application or a web application accessible by a mobile device such as a phone or tablet. The mobile device can be used, for example, in emergency medicine settings. In one embodiment, the method 100 can be executed using an application or a web application on a computer. The application can collect the age and weight of a patient in step 105. The application can then present an image of a segment of the body to the user in step 110. The segment of the body can be isolated from the rest of the body to improve the ease of collecting burn information for each segment individually. The application can collect digital input of burn information for the segment in step 115. The digital input of burn information can include a shading or selection of burned portions of each segment of the body. The presentation of each segment individually can enable a provider to accurately shade in the segment to match the location and size of patient burns. The burn information can be used to calculate the percentage of the segment that is burned in step 120. The burn percentage of each segment can be calculated individually. Each segment of the body makes up a portion (percentage) of the total surface area of a patient's body. The percentages can be stored in a look-up table corresponding to the patient's age and weight. The percentage of total surface area of the body segment can be determined using the look-up table in step 125. The percentage of total surface area of the burn on the body segment can then be calculated in step 130 by multiplying the burn percentage by the percentage of total surface area comprised by the body segment. The process of steps 110-130 can be repeated for all burned body segments. The percentage of total surface area of burns on each body segment can then be summed to determine the burn TBSA, or the percentage of the whole body that is affected by burns, in step 135.

Presenting the segments of the body individually is a significant improvement over existing methods of representing burns on a full view of the body. It is difficult to accurately depict burns on a full-body view, especially for smaller burns or burns that do not uniformly cover a segment of the body. Presenting each segment individually enables a provider to focus on the segment and ensure that the depiction of the burn matches the actual appearance of the burn. In one embodiment, each segment can be presented in sequence, e.g., from one end of the body to the opposite end of the body. Each segment can be presented according to an intended view (e.g., a posterior view, an anterior view). In another embodiment, a provider can input a selection of a desired segment of the body to color. The segments can be labeled using anatomical terms familiar to medical providers. In one embodiment, the segments can be presented with realistic proportions and scales. For example, the chest and abdomen can be presented as a segment of the body. The shoulders are usually wider than the waist, and an average or expected ratio between the shoulders and the waist can be presented in the image of the segment. In one embodiment, the relative sizes of different segments can be preserved in the presentation of the segments. The relative sizes can vary depending on the age and weight of the patient, as previously discussed. For example, the length of the head relative to the torso can be preserved in the presentation of the head and the torso for shading. In another embodiment, each segment can be presented to maximize the visibility of the segment in the display.

Burn information can include a selection, a representation, and/or an image of burns on a patient. In one embodiment, the burn information can be collected by presenting an image of each segment of the body. The image can be presented on a device display, such as a phone screen. The device can preferably be configured to accept user input, e.g., via a touchscreen, to collect burn information. In one embodiment, each image of a segment can be colored in or shaded to represent burns on a patient. The segment of the body is isolated in the image such that the image can be colored in to match the appearance of the segment on the body of the patient for greater accuracy. In one embodiment, visible features of the segment of the body can be illustrated in the image. For example, an image of the upper arm can include the elbow and part of the shoulder. These features can be used as visual landmarks to orient the image and can also provide indications of scale. In one embodiment, the image can be rotated within the plane of the display and/or scaled for more accurate shading. In one embodiment, the segment can be colored in according to a depth or degree (thickness) of the burn. For example, a first type of shading can be used to represent a second degree burn, while a second type of shading can be used to represent a third degree burn. A user can toggle between different types of shading. The burn TBSA can be calculated for all burns or for each burn depth individually.

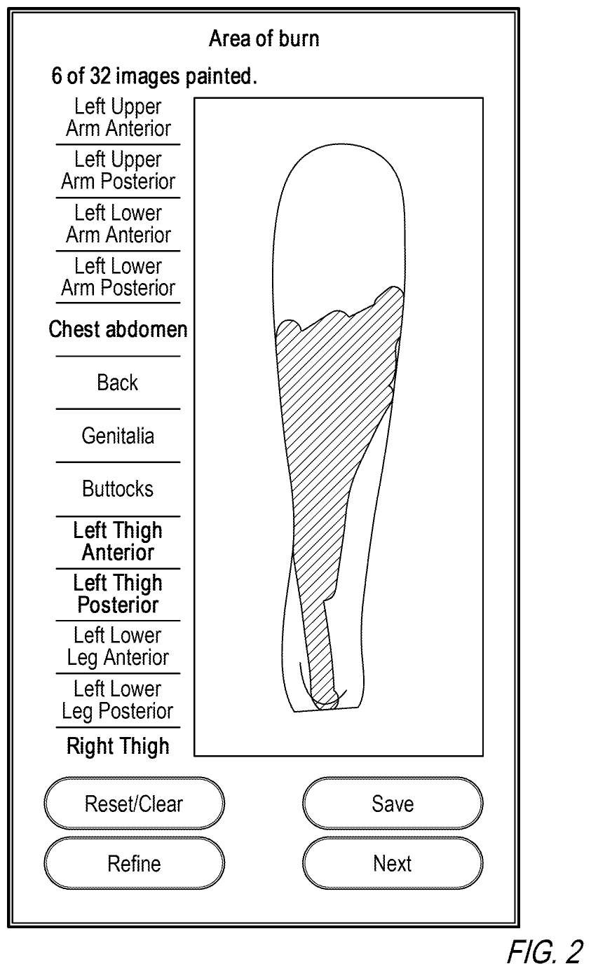

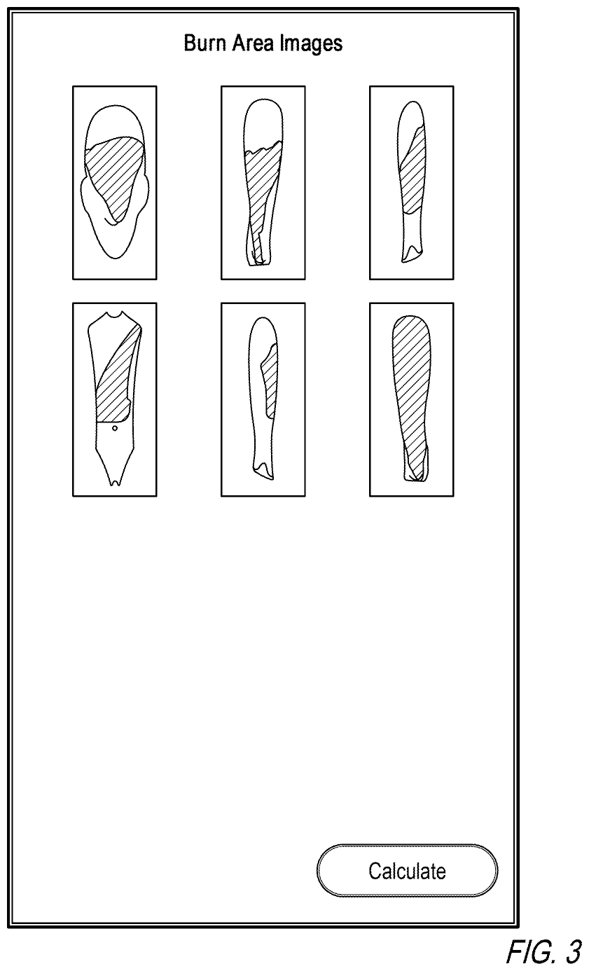

FIG. 2 illustrates an example of a segment of the body shaded to represent a burn. The segment of the body in this example is an anterior view of a thigh. The shape and proportions of the thigh are preserved in the image to clearly represent the thigh. Any portion of the segment can be shaded in to represent a burn on the thigh. The shading can also be removed. A list of segments can be presented in addition to the current segment to indicate progress across the entire body, as can be seen in FIG. 2. In one embodiment, a user can select the segment or segments that they will shade in. FIG. 3 illustrates an example of multiple segments of the body, including the head, the chest, the right thigh anterior, the right thigh posterior, the left thigh anterior, and the left thigh posterior. Each image can be shaded to represent burns on that portion of the patient's body. The segments enable accurate depiction of burns that span across various areas of the body. For example, as can be seen in FIG. 3, a burn can cover the majority of the anterior of a thigh and a smaller area of the posterior of the thigh.

In one embodiment, the burn information can include at least one image of at least one segment of the patient's body. In one embodiment, the image can be used to confirm the shaded segment. For example, the image can be overlaid onto the segment to verify that the shaded portions of the segment capture the burn on the body. The image can be adjusted (e.g., scaled) to match the illustration of the segment presented to the user. In another embodiment, the image can be analyzed, e.g., using computer vision, to determine what portion or percentage of the segment of the patient's body is burned. According to another embodiment, the image can be analyzed to determine a depth of the burn on the patient's body.

In one embodiment, how much of each segment is burned can be determined based on the burn information. The burn can be represented as a percentage of the surface area of the segment. For example, in FIG. 3, approximately half of the surface area of the head is shaded in to represent a burn. In one embodiment, the burn percentage for each segment can be calculated using a pixel-by-pixel calculation. For example, the burn percentage can be the shaded pixels as a proportion of the total number of pixels comprising the segment of the body. In one embodiment, the number of shaded pixels can be determined by examining the color information (e.g., RGB values) of each pixel in the image. In one embodiment, the number of shaded pixels can be determined using an image histogram. For example, the image of the body segment can be a uniform color (e.g., white) and intensity, while the shaded burn regions can be another color that will result in a separate peak on an intensity histogram. The number of pixels in the separate peak can be used to determine the burned percentage of the segment. In one embodiment, the pixels shaded for first-degree burns, second-degree burns, and third-degree burns can be distinguished from each other. In one embodiment, the method for determining the burn percentage can include dividing pixels into fractions. For example, the boundary of a segment of the body may include only a portion of a pixel. Additionally, a shaded burn region may only include a portion of a pixel or a plurality of pixels. Dividing a pixel into fractions can result in a more accurate burn percentage. In one embodiment, shading outside of an outline of the segment of the body can be removed or disregarded automatically. In one embodiment, the burn percentage can be determined as described above using an image of the patient burn in addition to or in place of the shaded segment.

The burn information can be used to determine a burn TBSA for a patient given the age and weight of the patient. The age and the weight of the patient can be collected in addition to the burn information. The combination of the age and the weight of the patient can be used to determine which look-up table or calculation method to use to determine burn TBSA. In one embodiment, the method can use approximately 10-40 age-weight delineations, wherein each age-weight delineation is a combination of an age and a weight or weight range. Each look-up table for distribution of body surface area can correspond to an age-weight delineation. In this manner, the look-up table can account for how a patient's body changes with age and for variations in weight for a given age. In one embodiment, a look-up table can be created using an expected weight or weight range for a patient's age. However, additional look-up tables can cover weights outside of the expected range for an age. The use of weight in addition to age provides more accurate estimations of body composition and surface area distribution than can be obtained with only age. For example, the present method can use a different look-up table for infant patients of 1 month, 2 months, 3 months, etc. In an example implementation, the present method can use a different look-up table for each month of an infant's development up until the infant is two years old. The infant's body and distribution of body surface area can change with each month. The present method can thus provide greater detail and accuracy than typical methods for assessing pediatric burns.

The burn TBSA of a patient can be determined using the burn percentages of each body segment and the distribution of total body surface area of the patient, as quantified in a look-up table. The burn percentage of each body segment can be multiplied by the proportion of total body surface area comprised by the body segment to determine the burn percentage of total body surface area that can be attributed to the burn on the body segment. The calculation can be repeated for each segment of the body, and the percentage of each segment can be added to determine the final burn TBSA. As an illustrative example, a patient can be burned at the anterior of the head and the anterior of a hand. The anterior of the head can comprise approximately 9% of total body surface area in an adult. If 50% of the anterior of the head is shaded to represent a burn, the burn on the anterior of the head encompasses approximately 4.5% of total body surface area. The anterior hand can comprise approximately 1.5% of total body surface area in an adult. If three-quarters of the hand (75%) are shaded to represent a burn, the burn on the anterior of the hand encompasses approximately 1.125% of the total body surface area. The total burn TBSA of the patient is the sum of the surface areas of the burn on the anterior of the head and the burn on the anterior of the hand, or 5.625%.

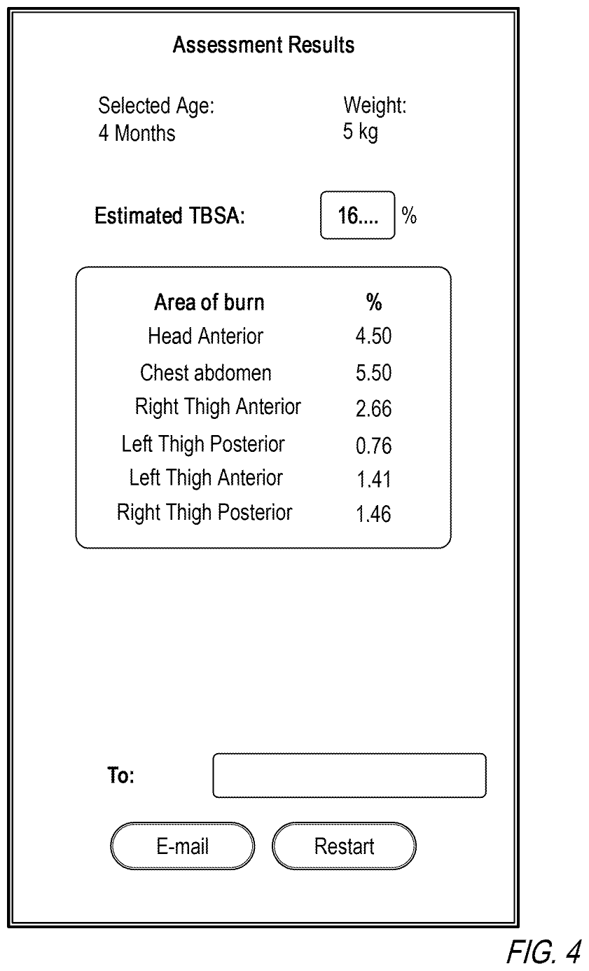

FIG. 4 illustrates an example of a burn TBSA estimation with a breakdown of burn percentages by segment. The age and weight of the patient are used in calculating the burn TBSA. According to the example, approximately 16% of the total body surface area is burned. The distribution of the 16% burn TBSA across the body segments can be presented, as illustrated in FIG. 4. For example, burns on the anterior of the head make up approximately 4.5% of the TBSA. The distribution of burn surface area can be presented as a percentage of the TBSA, as in FIG. 4. In another embodiment, the distribution of burn surface area can be presented as a percentage of the segment surface area rather than the total body surface area. The distribution of burn surface area can be used to determine which segments of the body contribute the most to the patient's burn and to inform further medical care. The results illustrated in FIG. 4 are calculated using the specific distribution of body surface area for a patient of the selected age and weight. For example, the results can be calculated using a look-up table for segments of the body of infant patients at 4 months old weighing 5 kilograms. Thus, the results are a more accurate assessment of burn TBSA than would be calculated using conventional methods that do not use age and weight or body segments.

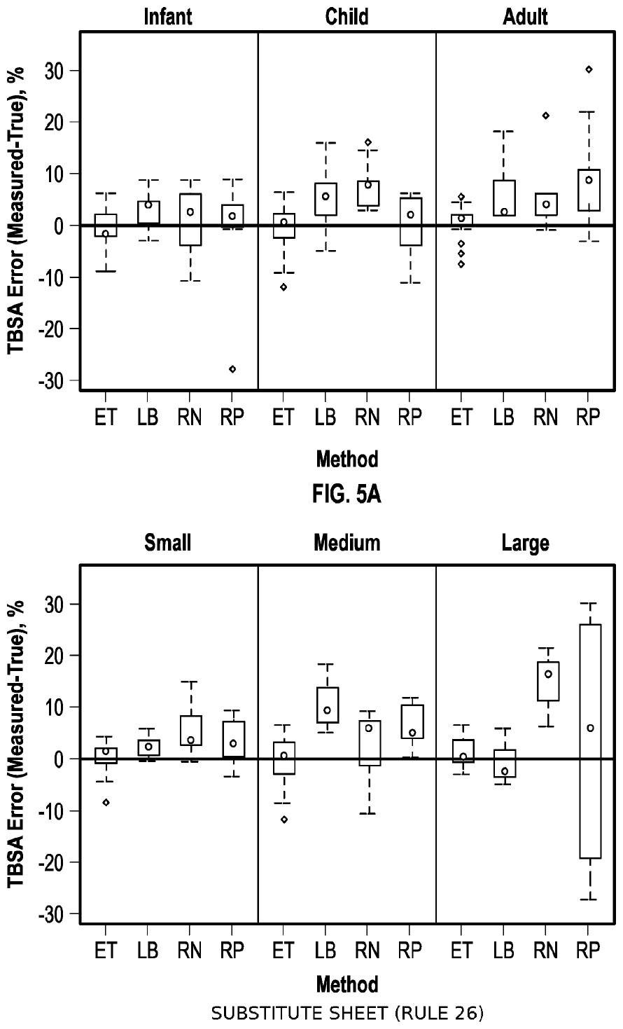

The systems and methods for determining burn TBSA can be used for patients of all ages and sizes as well as for small, medium, and large burns. FIG. 5 illustrates the accuracy of an exemplary implementation of the systems and methods disclosed herein (ET) compared with conventional methods such as the Lund-Browder Chart (LB), the Rule of Nines (RN), and the Rule of Palms (RP). The estimations used for the graphs of FIG. 5 were obtained by healthcare providers who did not specialize in burn treatment. The estimations of each method were compared with an actual burn TBSA on models, as determined by three-dimensional scanning and modeling. The method described herein was more accurate (smaller % error) than conventional methods in estimating burn TBSA for adult and child patients. The method described herein also resulted in a small average % error (mean of −0.35%) in estimating burn TBSA for infant patients. As illustrated in FIG. 5, the method described herein was more accurate than conventional methods in estimating small burn TBSA (e.g., <20% of TBSA) and medium burn TBSA (e.g., <20% to 49% of TBSA). Accurate methods for estimating small and medium-sized burns are important for triage purposes in determining how burn patients should be treated. Large burns are typically obvious upon visual inspection, and patients with large burn TBSA are immediately sent to burn treatment centers for fluid resuscitation and similar treatments. However, determining exact burn TBSA percentages is necessary for distinguishing small- and medium-sized burns in order to provide the right treatment and prevent the risks of overestimation or underestimation.

In one embodiment, the method can further include providing treatment recommendation. For example, a patient's burn TBSA can be compared to a threshold value to determine whether the patient should be transferred to a burn treatment center. In one embodiment, the threshold value can depend on the patient's age or the depth of the burns. In another embodiment, the treatment recommendation can include fluid resuscitation, wound dressings, therapy, or surgery. In one embodiment, the treatment recommendation can be based on how burns are distributed across the body. In one embodiment, the treatment recommendation can be based on additional medical information regarding the patient. In one embodiment, the method can include transmitting data (e.g., the images of the segments, the burn information, the burn TBSA data). The data can be transmitted to a user (e.g., via e-mail) or to a medical center. In one embodiment, the data can be used to update a patient record.

Embodiments of the subject matter and the functional operations described in this specification can be implemented by digital electronic circuitry, in tangibly embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of data processing apparatus. The computer storage medium can be a machine-readable storage device, a machine-readable storage substrate, a random or serial access memory device, or a combination of one or more of them.

The term “data processing apparatus” refers to data processing hardware and may encompass all kinds of apparatus, devices, and machines for processing data, including by way of example a programmable processor, a computer, or multiple processors or computers. The apparatus can also be or further include special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, Subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry. e.g., an FPGA an ASIC.

Computers suitable for the execution of a computer program include, by way of example, general or special purpose microprocessors or both, or any other kind of central processing unit. Generally, a CPU will receive instructions and data from a read-only memory or a random access memory or both. Elements of a computer are a CPU for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few. Computer-readable media suitable for storing computer program instructions and data include all forms of non-volatile memory, media and memory devices, including by way of example semiconductor memory devices, e.g., EPROM, EEPROM, and flash memory devices; magnetic disks. e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more Such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients (user devices) and servers. A client and server are generally remote from each other and typically interact through a communication network. The relationship of client and server arises by virtue of computer programs running on the respective computers and having a client-server relationship to each other. In an embodiment, a server transmits data, e.g., an HTML page, to a user device, e.g., for purposes of displaying data to and receiving user input from a user interacting with the user device, which acts as a client. Data generated at the user device, e.g., a result of the user interaction, can be received from the user device at the server.

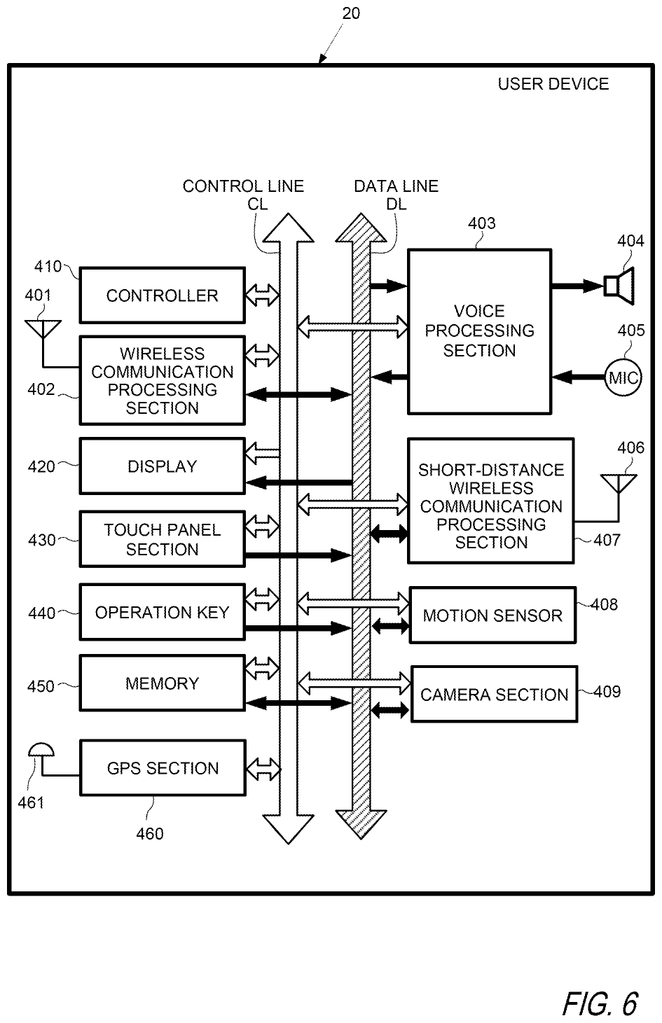

In an embodiment, the electronic user device 20 may be a smartphone. However, the skilled artisan will appreciate that the features described herein may be adapted to be implemented on other devices (e.g., a laptop, a tablet, a server, an e-reader, a camera, a navigation device, etc.). The exemplary user device 20 of FIG. 6 includes processing circuitry, as discussed above. The processing circuitry includes one or more of the elements discussed next with reference to FIG. 6. The electronic user device 20 may include other components not explicitly illustrated in FIG. 6 such as a CPU, GPU, frame buffer, etc. The electronic user device 20 includes a controller 410 and a wireless communication processor 402 connected to an antenna 401. A speaker 404 and a microphone 405 are connected to a voice processor 403.

The controller 410 may include one or more processors/processing circuitry (CPU, GPU, or other circuitry) and may control each element in the user device 20 to perform functions related to communication control, audio signal processing, graphics processing, control for the audio signal processing, still and moving image processing and control, and other kinds of signal processing. The controller 410 may perform these functions by executing instructions stored in a memory 450. Alternatively or in addition to the local storage of the memory 450, the functions may be executed using instructions stored on an external device accessed on a network or on a non-transitory computer readable medium.

The memory 450 includes but is not limited to Read Only Memory (ROM), Random Access Memory (RAM), or a memory array including a combination of volatile and non-volatile memory units. The memory 450 may be utilized as working memory by the controller 410 while executing the processes and algorithms of the present disclosure. Additionally, the memory 450 may be used for long-term storage, e.g., of image data and information related thereto.

The user device 20 includes a control line CL and data line DL as internal communication bus lines. Control data to/from the controller 410 may be transmitted through the control line CL. The data line DL may be used for transmission of voice data, displayed data, etc.

The antenna 401 transmits/receives electromagnetic wave signals between base stations for performing radio-based communication, such as the various forms of cellular telephone communication. The wireless communication processor 402 controls the communication performed between the user device 20 and other external devices via the antenna 401. For example, the wireless communication processor 402 may control communication between base stations for cellular phone communication.

The speaker 404 emits an audio signal corresponding to audio data supplied from the voice processor 403. The microphone 405 detects surrounding audio and converts the detected audio into an audio signal. The audio signal may then be output to the voice processor 403 for further processing. The voice processor 403 demodulates and/or decodes the audio data read from the memory 450 or audio data received by the wireless communication processor 402 and/or a short-distance wireless communication processor 407. Additionally, the voice processor 403 may decode audio signals obtained by the microphone 405.

The exemplary user device 20 may also include a display 420, a touch panel 430, an operation key 440, and a short-distance communication processor 407 connected to an antenna 406. The display 420 may be a Liquid Crystal Display (LCD), an organic electroluminescence display panel, or another display screen technology. In addition to displaying still and moving image data, the display 420 may display operational inputs, such as numbers or icons which may be used for control of the user device 20. The display 420 may additionally display a GUI for a user to control aspects of the user device 20 and/or other devices. Further, the display 420 may display characters and images received by the user device 20 and/or stored in the memory 450 or accessed from an external device on a network. For example, the user device 20 may access a network such as the Internet and display text and/or images transmitted from a Web server.

The touch panel 430 may include a physical touch panel display screen and a touch panel driver. The touch panel 430 may include one or more touch sensors for detecting an input operation on an operation surface of the touch panel display screen. The touch panel 430 also detects a touch shape and a touch area. Used herein, the phrase “touch operation” refers to an input operation performed by touching an operation surface of the touch panel display with an instruction object, such as a finger, thumb, or stylus-type instrument. In the case where a stylus or the like is used in a touch operation, the stylus may include a conductive material at least at the tip of the stylus such that the sensors included in the touch panel 430 may detect when the stylus approaches/contacts the operation surface of the touch panel display (similar to the case in which a finger is used for the touch operation).

In certain aspects of the present disclosure, the touch panel 430 may be disposed adjacent to the display 420 (e.g., laminated) or may be formed integrally with the display 420. For simplicity, the present disclosure assumes the touch panel 430 is formed integrally with the display 420 and therefore, examples discussed herein may describe touch operations being performed on the surface of the display 420 rather than the touch panel 430. However, the skilled artisan will appreciate that this is not limiting.

For simplicity, the present disclosure assumes the touch panel 430 is a capacitance-type touch panel technology. However, it should be appreciated that aspects of the present disclosure may easily be applied to other touch panel types (e.g., resistance-type touch panels) with alternate structures. In certain aspects of the present disclosure, the touch panel 430 may include transparent electrode touch sensors arranged in the X-Y direction on the surface of transparent sensor glass.

The touch panel driver may be included in the touch panel 430 for control processing related to the touch panel 430, such as scanning control. For example, the touch panel driver may scan each sensor in an electrostatic capacitance transparent electrode pattern in the X-direction and Y-direction and detect the electrostatic capacitance value of each sensor to determine when a touch operation is performed. The touch panel driver may output a coordinate and corresponding electrostatic capacitance value for each sensor. The touch panel driver may also output a sensor identifier that may be mapped to a coordinate on the touch panel display screen. Additionally, the touch panel driver and touch panel sensors may detect when an instruction object, such as a finger is within a predetermined distance from an operation surface of the touch panel display screen. That is, the instruction object does not necessarily need to directly contact the operation surface of the touch panel display screen for touch sensors to detect the instruction object and perform processing described herein. For example, in an embodiment, the touch panel 430 may detect a position of a user's finger around an edge of the display panel 420 (e.g., gripping a protective case that surrounds the display/touch panel). Signals may be transmitted by the touch panel driver, e.g. in response to a detection of a touch operation, in response to a query from another element based on timed data exchange, etc.

The touch panel 430 and the display 420 may be surrounded by a protective casing, which may also enclose the other elements included in the user device 20. In an embodiment, a position of the user's fingers on the protective casing (but not directly on the surface of the display 420) may be detected by the touch panel 430 sensors. Accordingly, the controller 410 may perform display control processing described herein based on the detected position of the user's fingers gripping the casing. For example, an element in an interface may be moved to a new location within the interface (e.g., closer to one or more of the fingers) based on the detected finger position.

Further, in an embodiment, the controller 410 may be configured to detect which hand is holding the user device 20, based on the detected finger position. For example, the touch panel 430 sensors may detect a plurality of fingers on the left side of the user device 20 (e.g., on an edge of the display 420 or on the protective casing) and detect a single finger on the right side of the user device 20. In this exemplary scenario, the controller 410 may determine that the user is holding the user device 20 with his/her right hand because the detected grip pattern corresponds to an expected pattern when the user device 20 is held only with the right hand.

The operation key 440 may include one or more buttons or similar external control elements, which may generate an operation signal based on a detected input by the user. In addition to outputs from the touch panel 430, these operation signals may be supplied to the controller 410 for performing related processing and control. In certain aspects of the present disclosure, the processing and/or functions associated with external buttons and the like may be performed by the controller 410 in response to an input operation on the touch panel 430 display screen rather than the external button, key, etc. In this way, external buttons on the user device 20 may be eliminated in lieu of performing inputs via touch operations, thereby improving watertightness.

The antenna 406 may transmit/receive electromagnetic wave signals to/from other external apparatuses, and the short-distance wireless communication processor 407 may control the wireless communication performed between the other external apparatuses. Bluetooth, IEEE 802.11, and near-field communication (NFC) are non-limiting examples of wireless communication protocols that may be used for inter-device communication via the short-distance wireless communication processor 407.

The user device 20 may include a motion sensor 408. The motion sensor 408 may detect features of motion (i.e., one or more movements) of the user device 20. For example, the motion sensor 408 may include an accelerometer to detect acceleration, a gyroscope to detect angular velocity, a geomagnetic sensor to detect direction, a geo-location sensor to detect location, etc., or a combination thereof to detect motion of the user device 20. In an embodiment, the motion sensor 408 may generate a detection signal that includes data representing the detected motion. For example, the motion sensor 408 may determine a number of distinct movements in a motion (e.g., from start of the series of movements to the stop, within a predetermined time interval, etc.), a number of physical shocks on the user device 20 (e.g., a jarring, hitting, etc., of the electronic device), a speed and/or acceleration of the motion (instantaneous and/or temporal), or other motion features. The detected motion features may be included in the generated detection signal. The detection signal may be transmitted, e.g., to the controller 410, whereby further processing may be performed based on data included in the detection signal. The motion sensor 408 can work in conjunction with a Global Positioning System (GPS) section 460. The information of the present position detected by the GPS section 460 is transmitted to the controller 410. An antenna 461 is connected to the GPS section 460 for receiving and transmitting signals to and from a GPS satellite.

The user device 20 may include a camera section 409, which includes a lens and shutter for capturing photographs of the surroundings around the user device 20. In an embodiment, the camera section 409 captures surroundings of an opposite side of the user device 20 from the user. The images of the captured photographs can be displayed on the display panel 420. A memory section saves the captured photographs. The memory section may reside within the camera section 109 or it may be part of the memory 450. The camera section 409 can be a separate feature attached to the user device 20 or it can be a built-in camera feature.

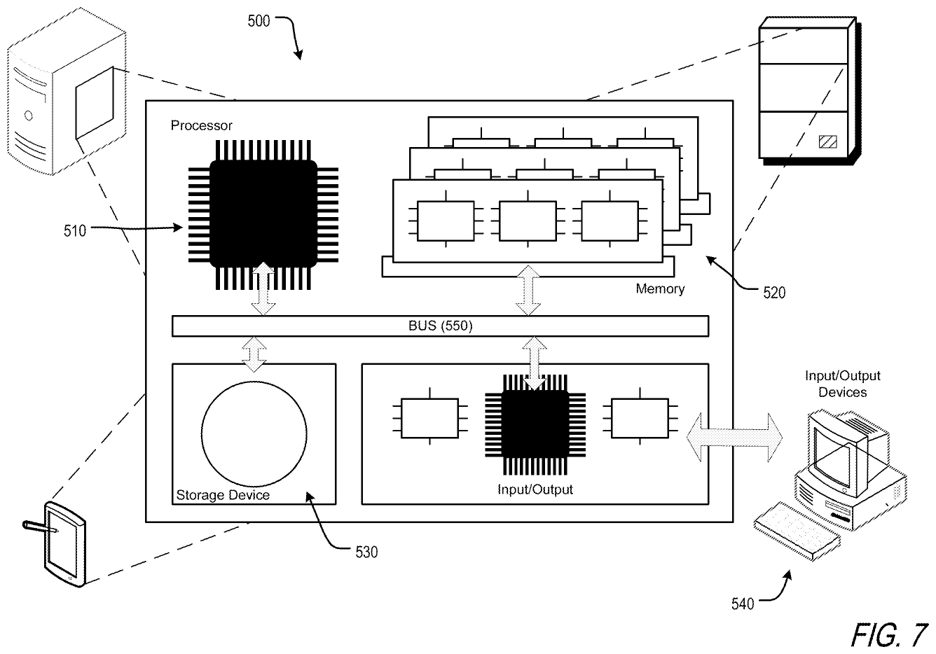

An example of a type of computer is shown in FIG. 7. The computer 500 can be used for the operations described in association with any of the computer-implement methods described previously, according to one implementation. The processing circuitry includes one or more of the elements discussed next with reference to FIG. 7. In FIG. 7, the computer 500 includes a processor 510, a memory 520, a storage device 530, and an input/output device 540. Each of the components 510, 520, 530, and 540 are interconnected using a system bus 550. The processor 510 is capable of processing instructions for execution within the system 500. In one implementation, the processor 510 is a single-threaded processor. In another implementation, the processor 510 is a multi-threaded processor. The processor 510 is capable of processing instructions stored in the memory 520 or on the storage device 530 to display graphical information for a user interface on the input/output device 540.

The memory 520 stores information within the computer 500. In one implementation, the memory 520 is a computer-readable medium. In one implementation, the memory 520 is a volatile memory unit. In another implementation, the memory 520 is a non-volatile memory unit.

The storage device 530 is capable of providing mass storage for the computer 500. In one implementation, the storage device 530 is a computer-readable medium. In various different implementations, the storage device 530 may be a floppy disk device, a hard disk device, an optical disk device, or a tape device.

The input/output device 540 provides input/output operations for the computer 500. In one implementation, the input/output device 540 includes a keyboard and/or pointing device. In another implementation, the input/output device 540 includes a display unit for displaying graphical user interfaces.

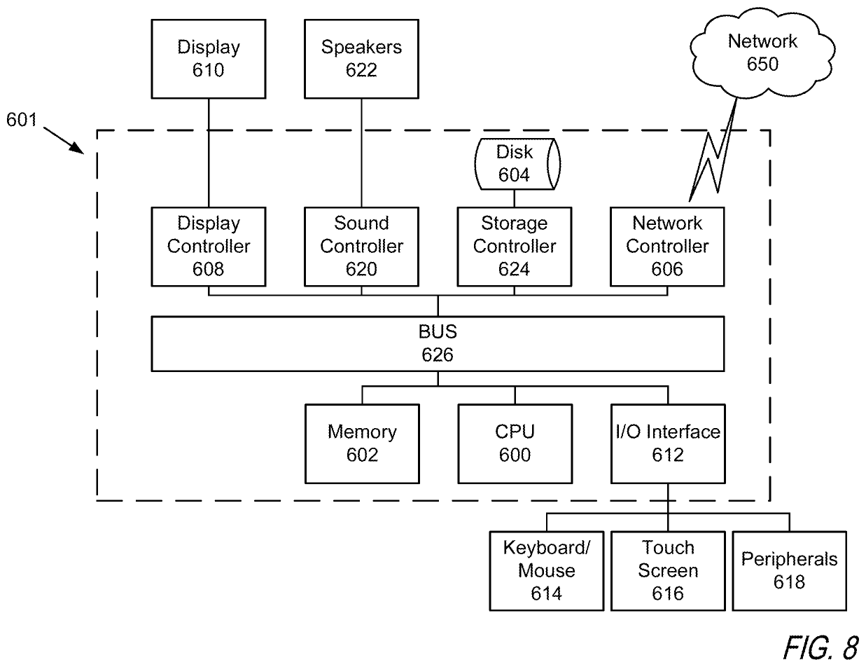

Next, a hardware description of a device 601 according to exemplary embodiments is described with reference to FIG. 8. In FIG. 8, the device 601 includes processing circuitry, as discussed above. The processing circuitry includes one or more of the elements discussed next with reference to FIG. 8. The process data and instructions may be stored in memory 602. These processes and instructions may also be stored on a storage medium disk 604 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the device 601 communicates, such as a server or computer.

Further, the claimed advancements may be provided as a utility application, background daemon, or component of an operating system, or combination thereof, executing in conjunction with CPU 600 and an operating system such as Microsoft Windows, UNIX, Solaris, LINUX, Apple MAC-OS and other systems known to those skilled in the art.

The hardware elements in order to achieve the device 601 may be realized by various circuitry elements, known to those skilled in the art. For example, CPU 600 may be a Xenon or Core processor from Intel of America or an Opteron processor from AMD of America or may be other processor types that would be recognized by one of ordinary skill in the art. Alternatively, the CPU 600 may be implemented on an FPGA, ASIC, PLD or using discrete logic circuits, as one of ordinary skill in the art would recognize. Further, CPU 600 may be implemented as multiple processors cooperatively working in parallel to perform the instructions of the processes described above.

The device 601 in FIG. 8 also includes a network controller 606, such as an Intel Ethernet PRO network interface card from Intel Corporation of America, for interfacing with network 650, and to communicate with other devices. As can be appreciated, the network 650 can be a public network, such as the Internet, or a private network such as an LAN or WAN network, or any combination thereof and can also include PSTN or ISDN sub-networks. The network 650 can also be wired, such as an Ethernet network, or can be wireless such as a cellular network including EDGE, 3G, 4G and 5G wireless cellular systems. The wireless network can also be WiFi, Bluetooth, or any other wireless form of communication that is known.

The device 601 further includes a display controller 608, such as a NVIDIA GeForce GTX or Quadro graphics adaptor from NVIDIA Corporation of America for interfacing with display 610, such as an LCD monitor. A general purpose I/O interface 612 interfaces with a keyboard and/or mouse 614 as well as a touch screen panel 616 on or separate from display 610. General purpose I/O interface also connects to a variety of peripherals 618 including printers and scanners.

A sound controller 620 is also provided in the device 601 to interface with speakers/microphone 622 thereby providing sounds and/or music.

The general purpose storage controller 624 connects the storage medium disk 604 with communication bus 626, which may be an ISA, EISA, VESA, PCI, or similar, for interconnecting all of the components of the device 601. A description of the general features and functionality of the display 610, keyboard and/or mouse 614, as well as the display controller 608, storage controller 624, network controller 606, sound controller 620, and general purpose I/O interface 612 is omitted herein for brevity as these features are known.

1. A method for determining a burn percentage of a patient, the method comprising:
presenting, via processing circuitry, an image of at least one segment of a body;
receiving, via the processing circuitry, a burn indication representing areas within the at least one segment of the body that have sustained a burn;
determining, via the processing circuitry, a burned percentage of the at least one segment of the body based on the burn indication;
determining, via the processing circuitry, a surface area of the at least one segment of the body based on an age of the patient and a weight of the patient; and
determining, via the processing circuitry, the burn percentage of the patient based on the burned percentage of the at least one segment of the body and the surface area of the at least one segment of the body.

2. The method of claim 1, wherein the at least one segment of the body is an anterior view or a posterior view.

3. The method of claim 1, wherein the burn indication includes an illustration of the burn on the image of the at least one segment of the body.

4. The method of claim 1, wherein the burn indication includes an image of the burn on the patient.

5. The method of claim 1, wherein the surface area of the at least one segment of the body is determined via a look-up table.

6. The method of claim 5, wherein the look-up table includes a distribution of body surface area based on the age of the patient and the weight of the patient.

7. The method of claim 6, wherein the age of the patient is an age in months.

8. An apparatus for determining a burn percentage of a patient, comprising:
processing circuitry configured to:
present an image of at least one segment of a body;
receive a burn indication representing areas within the at least one segment of the body that have sustained a burn;
determine a burned percentage of the at least one segment of the body based on the burn indication;
determine a surface area of the at least one segment of the body based on an age of the patient and a weight of the patient; and
determine the burn percentage of the patient based on the burned percentage of the at least one segment of the body and the surface area of the at least one segment of the body.

9. The apparatus of claim 8, wherein the at least one segment of the body is an anterior view or a posterior view.

10. The apparatus of claim 8, wherein the burn indication includes an illustration of the burn on the image of the at least one segment of the body.

11. The apparatus of claim 8, wherein the burn indication includes an image of the burn on the patient.

12. The apparatus of claim 8, wherein the surface area of the at least one segment of the body is determined via a look-up table.

13. The apparatus of claim 12, wherein the look-up table includes a distribution of body surface area based on the age of the patient and the weight of the patient.

14. The apparatus of claim 13, wherein the age of the patient is an age in months.

15. A non-transitory computer-readable storage medium for storing computer-readable instructions that, when executed by a computer, cause the computer to perform a method for determining a burn percentage of a patient, the method comprising:
presenting, via processing circuitry, an image of at least one segment of a body;
receiving, via the processing circuitry, a burn indication representing areas within the at least one segment of the body that have sustained a burn;
determining, via the processing circuitry, a burned percentage of the at least one segment of the body based on the burn indication;
determining, via the processing circuitry, a surface area of the at least one segment of the body based on an age of the patient and a weight of the patient; and
determining, via the processing circuitry, the burn percentage of the patient based on the burned percentage of the at least one segment of the body and the surface area of the at least one segment of the body.

16. The non-transitory computer-readable storage medium of claim 15, wherein the at least one segment of the body is an anterior view or a posterior view.

17. The non-transitory computer-readable storage medium of claim 15, wherein the burn indication includes an illustration of the burn on the image of the at least one segment of the body.

18. The non-transitory computer-readable storage medium of claim 15, wherein the burn indication includes an image of the burn on the patient.

19. The non-transitory computer-readable storage medium of claim 15, wherein the surface area of the at least one segment of the body is determined via a look-up table.

20. The non-transitory computer-readable storage medium of claim 19, wherein the look-up table includes a distribution of body surface area based on the age of the patient and the weight of the patient.
